(19) United States
(12) Patent Application Publication
(10) Pub. No.: US 2018/0187162 A1
(43) Pub. Date: Jul. 5, 2018

(54) VASCULARIZED TISSUE, SKIN OR MUCOSA EQUIVALENT

(71) Applicant: National University of Singapore, Singapore (SG)

(72) Inventors: Tong CAO, Singapore (SG); Harish Kiran HANDRAL, Singapore (SG); Sriram GOPU, Singapore (SG)

(21) Appl. No.: 15/739,001

(22) PCT Filed: Jun. 21, 2016
(86) PCT No.: PCT/SG2016/050282

§ 371 Date: Dec. 21, 2017

(30) Foreign Application Priority Data
Jun. 22, 2015 (GB) 1510913.5

(51) Int. Cl. C12N 5/071; A61L 27/60; A61L 27/38; G01N 33/50

(52) U.S. Cl. CPC C12N 5/0698; A61L 27/60; A61L 27/3834; A61L 27/3808; G01N 33/5008; A61L 27/3813; A61L 27/3804; A61L 27/3891; A61L 27/3895; A61L 27/3826

(57) ABSTRACT

The disclosure relates to a method for the differentiation of stem cells to endothelial cells, vascular smooth muscle cells, fibroblasts and keratinocytes; their use in the production of a organotypic vascularized skin or mucosa model or composition; a method relating thereto; the use of the model or composition in the testing of pharmaceutical and/or cosmetic agents; and including therapeutic and cosmetic skin compositions developed or tested thereby.

### FIELD OF THE INVENTION

The disclosure relates to a method for the differentiation of stem cells to endothelial cells, vascular smooth muscle cells (and/or pericytes), fibroblasts and keratinocytes; their use in the production of an organotypic optionally vascularized tissue, skin, or mucosa equivalent or composition; a method relating thereto; the use of the equivalent or composition in the testing of pharmaceutical and/or cosmetic agents; and including therapeutic and cosmetic skin compositions developed or tested thereby.

### BACKGROUND OF THE INVENTION

Human skin is the first line of defense for internal organs against invasion of pathogens and microorganisms. Accordingly, the skin serves as a vital protective layer for human body against water loss, and potential exogenous mechanical and chemical hazards. The epithelial surface of skin and oral mucosa is a stratified squamous tissue consisting of cells tightly attached to each other and arranged in a number of distinct layers (basal, prickle cell, granular and keratinized layers). The outermost part of skin is composed of multi-layered differentiated keratinocytes to shape a self-keratinized structure, called the epidermis. The epidermis is combined with supportive underlying layers of fibroblast cells, called the dermis layer.

Due to disruption of skin barrier function by aging and disease, there is great interest in developing skin treatment products. Further, in this regard and given the intrinsic barrier function of the skin, effective topical delivery of therapeutic compounds requires penetration across the superficial permeability barrier of the tissue. Successful translation of new therapeutics requires the ability to evaluate test agents in realistic model systems for cutaneous and mucosal delivery. The development of an in vitro model or equivalent that can reproduce the appropriate mechanical and permeability characteristics of the normal tissue is critical to the formulation and delivery of therapeutic compounds and to study barrier properties of the protective surface of skin and oral mucosa, and represents an important tool for preclinical testing and for facilitating the translation of therapeutic compounds into clinical use.

Various skin models exist including ex vivo human tissue biopsies or surgical specimens to study permeability and barrier properties of skin and oral mucosa, but there are numerous difficulties associated therewith including ethical issues, supply and experimental variability. Additionally, animal studies whilst proving to be useful have numerous drawbacks for studying barrier properties due to inherent cross-species variability. There is also a desire to move away from animal testing of medicinal agents. Current in vitro organotypic models of keratinized stratified tissue may exhibit some of the structural characteristics observed in vivo but they are expensive, highly variable and do not reproduce the barrier properties of the parent tissue.

Alternatively, cell and tissue culture models can offer advantages in terms of availability of tissue, cost and safety. However, current cell culture monolayers do not show differentiation that accompanies skin tissue stratification in vivo and thus do not show the barrier properties of the normal tissue.

The growth of stratified, differentiated human epithelium to form organotypic 3D cultures potentially overcomes the disadvantages of cell monolayers. 3D culture systems are biochemically and physiologically more similar to in vivo tissue. However, in practice it has not proved easy to grow organ cultures that can effectively reproduce the barrier function of a normal skin explant. For example, measurements of permeability of organotypic skin cultures has shown permeability to a variety of compounds to be 3-100 fold greater than for normal skin (Robert et al, 1997; Garcia et al, 2002; Barai et al, 2008). Further, current techniques require unfavourable harvesting of skin biopsies through surgical processes from individuals and expansion of obtained cells in laboratory conditions to provide a sufficient number of cells for these models, which can result in loss of morphology and the functionality of these cells. Moreover, these techniques also require the use of animal-derived proteins (serum) which could preclude their clinical use and affect the reproducibility of the process depending on the batch of serum used; the use of cells from different donors which restricts the clinical utility of the technology due to issues relating to limited availability of cells, donor-donor variability and immunogenicity; the development of a microfluidic scaffold that involves a complex fabrication process; and the use of genetically modified cells which limits clinical utility.

Thus current models are both expensive and suffer from batch variability. These issues for full-thickness skin models worsen, since two different types of cells (i.e. dermal and epidermal) are desired in a full thickness skin models.

There is therefore an unmet need for a representative and reproducible organotypic skin model that faithfully recapitulates the features of human skin which can facilitate identification of therapeutic and cosmetic agents and research into skin disease.

This disclosure relates to an organotypic skin/mucosa tissue equivalent model or equivalent that is full-thickness, optionally but advantageously vascularized and authentically differentiated to provide an equivalent that is more representative i.e. morphologically and functionally of human tissue/skin. Moreover, the equivalent is made using material of known genetic origin—that is functionally stable and limits the introduction of adventitious infectious agents to provide superior stability and longevity compared to existing equivalents, with application in the screening, development and evaluation of the effectiveness of cosmetics, pharmaceutical agents, and therapeutics.

### STATEMENTS OF INVENTION

According to an aspect of the invention there is provided a method for the preparation of an organotypic vascularized tissue, skin or mucosa equivalent or composition comprising the steps of:
i) obtaining a preparation of mammalian pluripotent stem cells and culturing the cells under cell culture conditions to induce the formation of the following differentiated cell types: endothelial cells (SC-ECs), vascular smooth muscle cells and/or pericytes (collectively termed SC-vSMCs), fibroblasts (SC-Fib) and keratinocytes (SC-KCs);
ii) seeding the SC-ECs, SC-vSMCs and, optionally, SC-Fib of part i) in or on a scaffold and further culturing the cells under cell culture conditions to induce the formation of a vascularized dermal layer;
iii) seeding the SC-KCs of part i) onto the vascularized dermal layer of part ii) and further culturing the cells under cell culture conditions to induce the formation of a stratified layer of keratinized epidermis upon said vascularized dermal layer to provide an organotypic vascularized skin or mucosa equivalent; and
iv) maintaining said organotypic vascularized skin or mucosa equivalent prepared by the steps of i)-iii) in cell culture.

In certain embodiments said keratinocytes are dermal keratinocytes (SC-KCs) and/or oral mucosal keratinocytes (SC-oral-KCs) and in the former instance where only dermal keratinocytes are used one obtains a dermal model and in the later instance where only oral keratinocytes are used one obtains an oral model.

In certain embodiments, said mammalian pluripotent stem cells are embryonic in origin, such as human, embryonic stem cells (hESC) or human embryonic germ cells (hEGC). Alternatively, or additionally, said mammalian pluripotent stem cells are induced pluripotent stem cells, such as, human induced pluripotent stem cells (hiPSC). Advantageously, this permits consistent epidermal and full-thickness skin or mucosa equivalents populated with dermal and epidermal cells with the requisite barrier properties to be generated by providing potentially an unlimited source of skin cells. Further, by incorporation of human hESC/hEGC/hiPSC-derived cell lines into skin equivalents (SE), they offer a more true reflection of the cellular phenotypes observed in vivo.

Reference herein to cell culture conditions includes reference to a medium designed to support the growth of cells according to the invention, in particular stem cells or cells derived therefrom. Many different types of chemical medium can be used to support the growth of stem or progenitor cells in culture or cells derived therefrom, such as but not limited to, feeder support system medium which is either supplemented with fetal bovine serum or serum replacer and feeder-free systems supplemented with defined culture media, such as mTeSR™1 and TeSR™8.

However, all cell cultures used in connection with the claimed method can optionally be serum-free cell cultures and also optionally feeder free (minimal use of animal-derived cells and proteins). In certain embodiments, a method where a serum-free medium is composed of basal medium supplemented with serum replacer and growth supplements in a feeder free system is utilized.

Further, in yet certain methods said cell culture medium comprises at least one other compound, agent, or drug useful in supporting normal cellular survival, metabolism or differentiation, such as but not limited to retinoic acid, vascular endothelial growth factor (VEGF), basic fibroblast growth factor (bFGF), epidermal growth factor (EGF), hydrocortisone, transferrin, ascorbic acid, calcium chloride, insulin, aprotinin, inhibitors of glycogen synthase-3 (that includes but not limited to CHIR99021, BIO, SB 216763, SB 415286, CHIR-98014) or bone morphogenetic proteins 4 (BMP4).

In certain methods, said cell culture conditions comprise additional cell types such as but not limited to melanocytes or macrophages. The co-culture of cells with melanocytes provides an epithelial skin equivalent exhibiting pigmentation, permitting assessment of the effects of UV exposure and anti-UV materials on the skin. Similarly again, use of macrophages permits development of an immunocompetent in vitro skin equivalent for testing immune sensitization of drugs and establishing in vitro disease equivalents; in certain embodiments, said additional cell types are autologous or derived from the stem cells.

Additionally, according to a certain methods, said additional cell types are derived from human embryonic stem cells (hESC).

In other methods, where iPSCs is practised, said cells are autologous and so the organotypic, ideally vascularised, skin or mucosa equivalent is bespoke for a particular person.

In certain other methods, said method comprises culturing said cells in step ii) for at least 1-20 days prior to step iii), or 2-14 days, or a number of days selected from the group comprising of: 2, 3, 4, 5, 6, 7, 8, 9, 10, 11, 12, 13, and 14 days.

In other methods, differentiation of said mammalian pluripotent stem cells to induce the formation of a differentiated cell type selected from the group comprising: endothelial cells (hESC-ECs), vascular smooth muscle cells cells and/or pericytes (collectively termed hESC-vSMCs), fibroblasts (hESC-Fib) and keratinocytes (hESC-KCs) comprises the use of cell culture media as set forth in the methods section described herein, in particular parts 1-4 thereof and/or methods as set forth in the methods section described herein, in particular parts 1-4 thereof, including the ranges described therein and in particular the typical amounts/concentrations/ratios used therein.

In other methods, said skin keratinocytes and oral keratinocytes are made by the use of the cell culture media and/or method described in parts 2 & 3 of the methods section, respectively, including the ranges described therein and in particular the typical amounts/concentrations/ratios used therein.

In further methods, seeding the SC-ECs, SC-vSMCs and, optionally, SC-Fib of part i) in or on a scaffold and further culturing the cells under cell culture conditions to induce the formation of a vascularized dermal layer comprises the use of cell culture media as set forth in the methods section described herein, in particular parts 6 & 7 thereof and/or methods as set forth in the methods section described herein, in particular parts 6 & 7 thereof, including the ranges described therein and in particular the typical amounts/concentrations/ratios used therein.

Reference herein to a scaffold refers to any material that is capable of supporting three-dimensional tissue cell culture by replicating an in vivo cellular environment including cell attachment, cellular signalling and diffusion and mechanical support. As will be appreciated by those skilled in the art, numerous different types of scaffolds exist and can be used in accordance with the method described herein such as cell culture scaffolds having the requisite porosity to facilitate cell seeding and diffusion throughout the whole structure of both cells and nutrients.

An example of a cell culture scaffold is disclosed in US2010/048411, the content of which is incorporated by reference. These substrates comprise microcellular polymeric materials which are described as “polyHIPE” polymers. These polymers form reticulate structures of pores that interconnect with one another to provide a substrate to which cells can attach and proliferate. The process for the formation of polyHIPEs allows pore volume to be accurately controlled with pore volume varying from 75% to 97%. Pore sizes can vary between 0.1 to 1000 micron and the diameter of the interconnecting members from a few microns to 100 microns. Furthermore the polyHIPEs can be combined with additional components that facilitate cell proliferation and/or differentiation. PolyHIPEs are therefore versatile substrates on which cells can attach and proliferate in a cell culture system. Processes for the preparation of polyHIPEs are well known in the art and also disclosed in WO2004/005355 and WO2004/004880. PolyHIPEs are commercially available and comprise for example oil phase monomers styrene, divinyl benzene and a surfactant, for example Span 80 sorbitan monooleate. In addition, the rigidity of the polymer formed during processing of the polyHIPE may be affected by the inclusion of a monomer such as 2-ethylhexyl acrylate. The process for the formation of polyHIPE from an emulsion is initiated by the addition of a catalyst such as ammonium per-sulphate.

In a certain methods, said scaffold comprises a biocompatible polymer based scaffold such as but not limited to a polyester including polystyrene, polylactic acid, polyglycolic acid, polycaprolactone, poly-dl-lactic-co-glycolic acid, or the like. The cell support substrate can be degradable or non-degradable.

In other methods, said scaffold is a fibrin-based scaffold, it advantageously overcomes the limitations associated with other published and commercially available skin equivalents such as shrinkage of the skin, short-term culture and lack of blood supply.

In other methods, said scaffold is a gel scaffold, such as but not limited to a polyethylene glycol-fibrin, fibrin, collagen type-I gel scaffold, of the like. The scaffold can be cultured in a cell culture media as set forth in the methods section described herein, in particular part 6 thereof and/or prepared as set forth in the methods section described herein, in particular part 6 thereof, including the ranges described therein and in particular the typical amounts/concentrations/ratios used therein.

In certain embodiments, the hESC-ECs, hESC-vSMCs and hESC-Fib are provided in a ratio of about 10:1:1 to about 40:1:1; about 10:1:1 to about 35:1:1; about 10:1:1 to about 30:1:1; about 10:1:1 to about 25:1:1; about 15:1:1 to about 25:1:1; about 17:1:1 to about 25:1:1; about 17:1:1 to about 22:1:1; about 18:1:1 to about 22:1:1; about 18:1:1 to about 21:1:1; or about 19:1:1 to about 21:1:1 in the scaffold. In certain embodiments, the hESC-ECs, hESC-vSMCs and hESC-Fib are provided in a ratio of about 20:1:1 in the scaffold. In certain embodiments, the scaffold is a PEG-fibrin gel scaffold.

In the examples below, the PEG-fibrin gel with the hESC-ECs, hESC-vSMCs and hESC-Fib were nourished with 3D vascularization media (described below) for 10 days with media changes every 24 hours. After the 10-day 3D tri-culture period step iii) above was undertaken.

In yet further methods, seeding the hESC-KCs of part i) onto the vascularized dermal layer of part ii) and further culturing the cells under cell culture conditions to induce the formation of a stratified layer of keratinized epidermis upon said vascularized dermal layer to provide an organotypic vascularized skin or mucosa equivalent comprises the use of serum-free cell culture media as set forth in the methods section described herein, in particular parts 7 & 8 thereof and/or the use of methods as set forth in the methods section described herein, in particular parts 7 & 8 thereof, including the ranges described therein and in particular the typical amounts/concentrations/ratios used therein.

In certain embodiments, the keratinocytes can be seeded on top of the vascularized dermal layer at a seeding density of about 10×10⁴ to about 40×10⁴; about 10×10⁴ to about 35×10⁴; about 10×10⁴ to about 30×10⁴; about 15×10⁴ to about 30×10⁴; about 20×10⁴ to about 30×10⁴; about 20×10⁴ to about 29×10⁴; about 21×10⁴ to about 29×10⁴; about 21×10⁴ to about 28×10⁴; about 22×10⁴ to about 28×10⁴; about 22×10⁴ to about 27×10⁴; about 23×10⁴ to about 27×10⁴; about 23×10⁴ to about 26×10⁴; or about 24×10⁴ to about 26×10⁴. In certain embodiments, the keratinocytes can be seeded on top of the vascularized dermal layer at a seeding density of 25×10⁴ cells/cm². For the generation of in vitro vascularized skin equivalent, hESC-KCs can be seeded, while for the generation of in vitro vascularized mucosa equivalent, hESC-oralKCs can be seeded. In this phase of keratinocyte culture, the PEG-fibrin gels were nourished with 3D epithelial media (described below) for 2-3 days with media renewed every 24 hours.

In other methods, said mammalian keratinocytes are cultured at an Air-Liquid Interface. This can be done by transferring a culture to a (for e.g. 12-well) deep well plate (Griener BioOne) and media supplied from only the bottom surface (while the top surface was exposed to air). The media, ideally, used at this phase can be 4 mL/well of 3D cornification media (described below). At the end of the third week of culture using an air-liquid interface the equivalent was finished.

Reference herein to the term Air-Liquid Interface (ALI) refers to the culture of cells such that their basal membrane is in contact with, or submerged in, liquid and their apical membrane is in contact with air. Advantageously, the keratinocytes consequently demonstrate apical-basal polarity in their differentiation resulting in the development of functional keratinised surfaces as seen in vivo.

According to a further aspect, there is provided an isolated differentiated endothelial cell (hESC-ECs), vascular smooth muscle cell and/or pericyte (collectively termed hESC-vSMCs), fibroblast (hESC-Fib) or keratinocyte, dermal or oral, (hESC-KCs) obtained or when obtained or obtainable by the method according to the invention.

According to a further aspect, there is provided an isolated organotypic vascularized tissue, skin or mucosa equivalent obtained or when obtained or obtainable by the method according to the invention.

According to a further aspect, there is provided a method for the preparation of an organotypic tissue or skin or mucosa equivalent or composition comprising the steps:
i) seeding endothelial cells and vascular smooth muscle cells/pericytes and, optionally, fibroblasts in or on a scaffold to provide a vascularized dermal layer;
ii) seeding keratinocytes onto the vascularized dermal layer of part i) and further culturing the cells under cell culture conditions to induce the formation of a stratified layer of keratinized epidermis upon said vascularized dermal layer to provide an organotypic skin or mucosa equivalent; and
iii) maintaining said organotypic tissue, skin or mucosa equivalent prepared by the steps of i)-ii) in cell culture.

In certain embodiments said keratinocytes are dermal keratinocytes (SC-KCs) and/or oral mucosal keratinocytes (SC-oral-KCs) and in the former instance where only dermal keratinocytes are used one obtains a dermal equivalent and in the later instance where only oral keratinocytes are used one obtains an oral equivalent.

In certain methods, said cells are autologous and so the organotypic tissue, skin or mucosa equivalent is bespoke for a particular person.

According to a further aspect, there is provided an organotypic tissue, skin or mucosa equivalent obtained or when obtained or obtainable by the either method according to the invention.

According to a further aspect, there is provided a therapeutic tissue/skin graft or implant comprising an organotypic skin composition obtained or when obtained or obtainable by either method according to the invention.

According to a yet further aspect of the invention there is provided an organotypic tissue/skin graft or implant according to the invention for use in the treatment of skin damage.

In certain embodiments, skin damage includes damage caused by infection or trauma, including wounding, scarring, or burns, or in response to disease such as skin grafts required as a consequence of tissue removal in cancer or in the treatment of diabetic or non-diabetic ulcers.

According to a further aspect, there is provided a cosmetic tissue/skin graft or implant comprising an organotypic skin composition obtained or obtainable by either method according to the invention.

According to a further aspect, there is provided a method of treatment comprising administering or implanting a tissue/skin graft or implant according to either method of the invention at or into a site of a mammal to be treated.

According to yet a further aspect, there is provided a method of cosmetic surgery comprising implanting a tissue/skin graft or implant according to either method of the invention into a site of a mammal to be treated.

According to a further aspect, there is provided a cell culture vessel comprising an organotypic tissue, skin or mucosa equivalent according to the invention.

In a certain embodiments, said cell culture vessel comprises a cell culture insert, optionally removable, containing said organotypic tissue, skin or mucosa equivalent and in fluid contact with cell culture medium.

In a certain embodiments, said culture vessel comprises cell culture media as set forth in the methods described herein.

According to a further aspect, there is provided an organotypic tissue, skin or mucosa equivalent according to the invention for use in the testing of test agents such as but not limited to therapeutics, drugs, dermal ointments, oral/dental products, cosmetics, compounds or biologically active xenobiotic agents, on skin cell function and permeability.

The term “xenobiotic agent” is herein given a broad definition and includes not only compounds but also gaseous agents. Typically, xenobiotic agent encompasses pharmaceutically active agents used in human and veterinary medicine and human cosmetics.

In yet a certain embodiments, said test agent can contact the cell culture by adding it to said cell culture medium. Alternatively, said test agent can contact the cell culture by adding it to the apical surface of said organotypic equivalent. Advantageously, this permits delivery of test agents, including vapours, gases and dry air-borne powders, in addition to soluble test-agents, this is much more representative of events that occur in-vivo wherein the skin epithelium is one of the first lines of defense to a variety of different agents.

According to a further aspect, there is provided a cell array wherein said array comprises a plurality of cell culture vessels according to the invention.

The screening of large numbers of agents requires preparing arrays of cells for the handling of cells and the administration of agents. Assay devices, for example, include standard multiwell micro-titre plates with formats such as 6, 12, 24 48, 96 and 384 wells which are typically used for compatibility with automated loading and robotic handling systems. Typically, high throughput screens use homogeneous mixtures of agents with an indicator compound which is either converted or modified resulting in the production of a signal. The signal is measured by suitable means (for example detection of fluorescence emission, optical density, or radioactivity) followed by integration of the signals from each well containing the cells, agent and indicator compound.

In certain embodiments, said mammalian keratinocytes are cultured at an Air-Liquid Interface.

According to a further aspect, there is provided a method for the high throughput screening of test agents comprising the steps:
i) providing an array according to the invention;
ii) contacting the array with a plurality of agents to be tested;
iii) collating activity data obtained following (ii) above;
iv) converting the collated data into a data analyzable form; and optionally
v) providing an output for the analysed data.

In certain methods, the organotypic equivalent is contacted with at least one therapeutic, cosmetic, compound or xenobiotic agent.

In certain methods, said mammalian keratinocytes are cultured at an Air-Liquid Interface.

The culture method can result in the advantageous formation of a stable dermal layer in the cell support substrate. Further, culture of keratinocytes upon said fibroblast/support substrate dermal layer at the Air-Liquid interface can lead to keratinocytes demonstrating apical-basal polarity in their differentiation resulting in the development of functional keratinised or non-keratinised surfaces with epidermal stratification as seen in vivo. Additionally, it has been found that without embedding fibroblasts within enclosed substrates cellular interactions between the skin layers can be explored. This therefore results in the formation of a reliable and realistic skin equivalent with superior stability and longevity which has application in reconstructive skin surgery.

Any further aspect may, in certain embodiments, include or be characterised by any of the aforementioned features.

As used herein, the term ‘about’ when used in connection with a numerical value means numerical values encompassing and including ±10%, ±9%, ±8%, ±7%, ±6%, ±5%, ±4%, ±3%, ±2%, ±1%, or ±0% of said numerical value.

Throughout the description and claims of this specification, the words “comprise” and “contain” and variations of the words, for example “comprising” and “comprises”, means “including but not limited to”, and is not intended to (and does not) exclude other moieties, additives, components, integers or steps.

Throughout the description and claims of this specification, the singular encompasses the plural unless the context otherwise requires. In particular, where the indefinite article is used, the specification is to be understood as contemplating plurality as well as singularity, unless the context requires otherwise.

Features, integers, characteristics, compounds, chemical moieties or groups described in conjunction with a particular aspect, embodiment or example of the invention are to be understood to be applicable to any other aspect, embodiment or example described herein unless incompatible therewith.

Moreover, unless stated otherwise, any feature disclosed herein may be replaced by an alternative feature serving the same or a similar purpose.

No admission is made that any reference referred to herein constitutes prior art. Further, no admission is made that any of the prior art constitutes part of the common general knowledge in the art.

An embodiment of the invention will now be described by example only and with reference to the following figures:

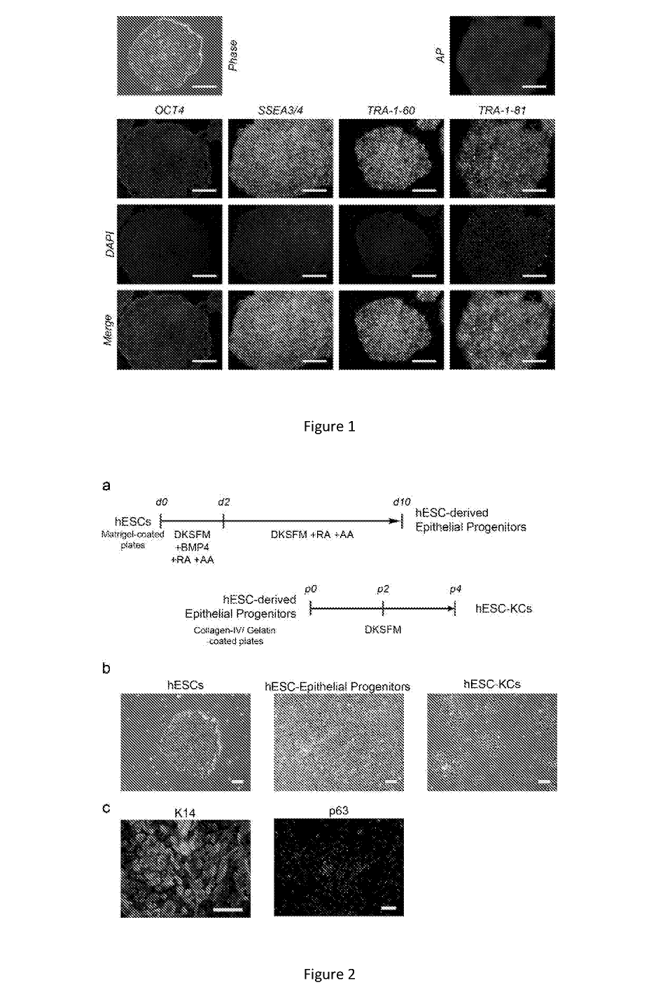

FIG. 1: Analysis of pluripotency status of hESCs cultured over Matrigel. Top left photomicrograph shows the compact, well defined morphology of hESC colony upon culture over Matrigel and mTeSR1. Immunofluorescence micrographs show the expression of pluripotency markers OCT4, SSEA4, TRA-1-60, TRA-1-81 and alkaline phosphatase (AP). Scale bars: 500 μm.

FIG. 2: (a) Schematic representation of differentiation of hESCs to hESC-derived epithelial progenitors by sequential treatment with BMP4, retinoic acid (RA) and ascorbic acid (AA) for 48 hours followed by RA and AA in defined keratinocyte serum-free medium (DKSFM). The hESC-derived epithelial progenitors were passaged onto collagen-IV (1 μg/cm²)/0.1% gelatin coated plates and propagated in DKSFM to yield hESC-KCs. (b) Representative photomicrographs showing the phase contrast images of hESCs, hESC-derived epithelial progenitors and hESC-KCs. (c) Representative photomicrographs showing immunofluorescent images of hESC-KCs stained for keratinocyte markers K14 and p63. Scale bar: in (b)-200 μm, in (c)-100 μm.

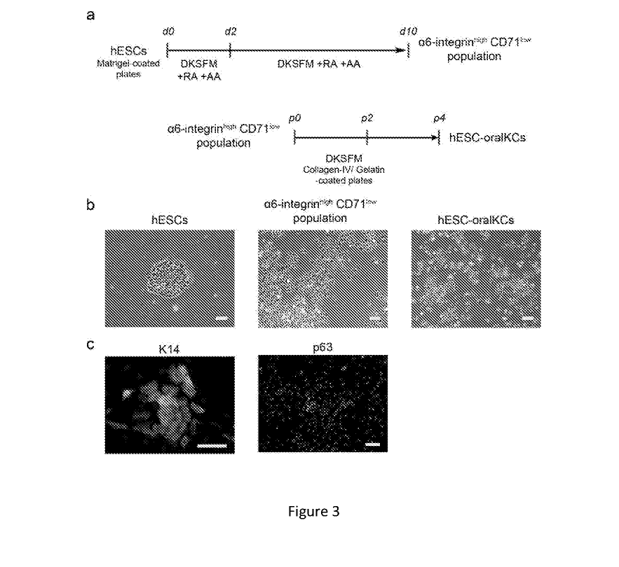

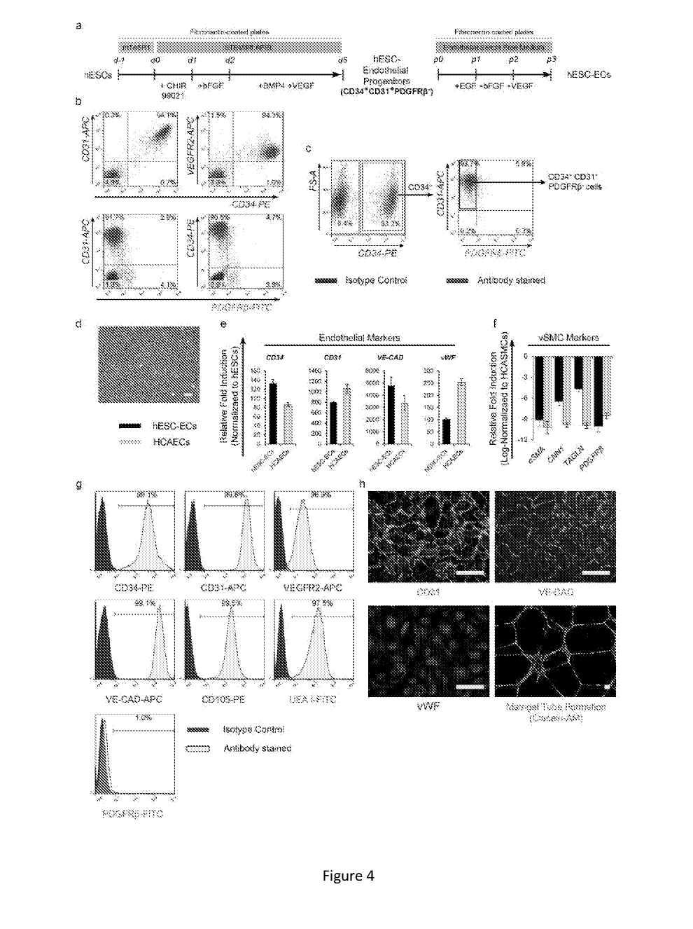

FIG. 3: (a) Schematic representation of differentiation of hESCs to hESC-derived epithelial progenitors by sequential treatment with retinoic acid (RA-1 μM) and ascorbic acid (AA-50 μg/ml) for 48 hours followed by RA (0.5 μM) and AA (50 μg/ml) in defined keratinocyte serum-free medium (DKSFM). FACS sorted α6-integrin^{high} and CD71^{low} population is passaged onto collagen-IV (1 μg/cm²)/0.1% gelatin coated plates and propagated in DKSFM to yield hESC-oralKCs. (b) Representative photomicrographs showing the phase contrast images of hESCs, α6-integrin^{high} and CD71^{low} population and hESC-oralKCs. (c) Representative photomicrographs showing immunofluorescent images of hESC-oralKCs stained for keratinocyte markers K14 and p63. Scale bar: in (b)-200 μm, in (c)-100 μm.

FIG. 4: (a) Schematic representation of differentiation of hESCs to hESC-endothelial progenitors (CD34+CD31+ cells) by sequential treatment with CHIR99021 (+GSKi), bFGF, and VEGF. The hESC-derived endothelial progenitors were sorted using flow cytometry after 5 days of differentiation and further differentiated towards hESC-ECs (b,c) Flow cytometry based sorting of hESC-endothelial progenitors for CD31+CD34+PDGFRβ-cells. (d) Photomicrograph shows the cobblestone morphology of hESC-ECs under phase contrast microscopy. Real time RT-PCR analysis of transcripts related to endothelial (e) and vSMC (f) lineages. (g) Flow cytometry histogram overlays showing the expression of endothelial lineage associated markers, binding to lectin UEA-I, and lack of PDGFRβ expression. (h) Immunofluorescent micrographs showing the surface expression of CD31 and VE-CAD, cytoplasmic expression of vWF and formation of tube-like structures on matrigel. Scale bars: 100 μm.

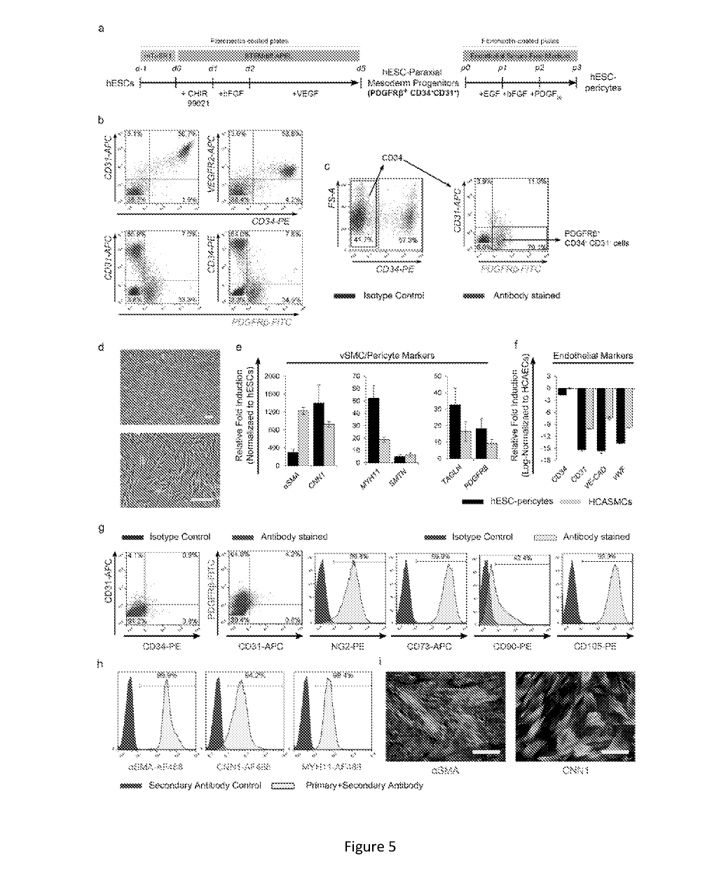

FIG. 5: (a) Schema for differentiation of hESCs to hESC-paraxial mesoderm progenitors and then to hESC-pericytes under feeder- and serum-free conditions. Representative flow cytometry overlays of: (b) expression of CD34, CD31, VEGFR2 and PDGFRβ; (c) co-expression of CD34, CD31 and PDGFRβ, and sorting of PDGFRβ+CD34-CD31− cells. (d) Phase contrast micrograph showing the spindle-shaped morphology of hESC-vSMCs. Real time RT-PCR analysis of transcripts related to vSMC/pericytes (e) and endothelial (f) lineages. (g) Flow cytometry overlays showing the expression of surface markers related to endothelial lineage (CD34, CD31), vSMC/pericyte lineage (PDGFRβ, NG2), and mesenchymal lineage (CD73, CD90, CD105). (h) Flow cytometry histogram overlays showing the expression of cytoplasmic cytoskeletal proteins related to vSMC lineage. (i) Immunofluorescent micrographs showing the cytoplasmic expression of aSMA and calponin (CNN1). Scale bars: 100 μm.

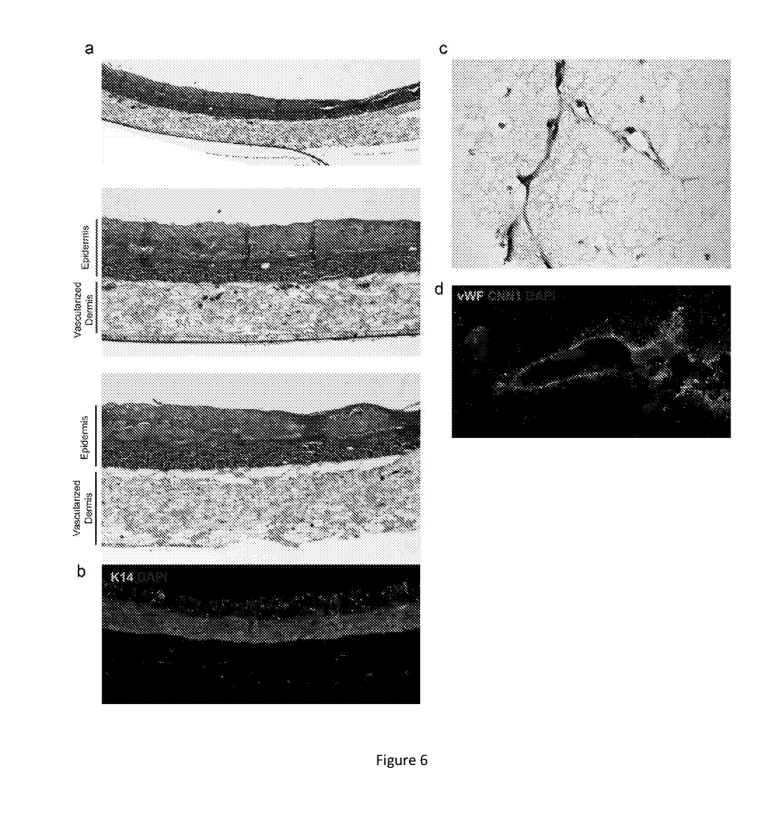

FIG. 6: (a) Representative photomicrographs of haematoxylin and eosin (H-E) stained sections of 3D in vitro vascularized skin. The epidermis consists of stratified layers of keratinocytes and cornification, while the dermis shows the presence of microvasculature and fibroblasts. (b) Immunofluorescent photomicrograph of formalin-fixed paraffin-embedded sections of 3D in vitro vascularized skin showing the expression of K14. (c) Photomicrographs of H-E stained sections of the dermal layer of 3D in vitro vascularized skin showing the presence of microvascular channels. (d) Immunofluorescent photomicrograph of formalin-fixed paraffin-embedded sections of 3D in vitro vascularized skin showing the presence of lumenized microvascular channels with expression of vWF and CNN1 by hESC-ECs and hESC-vSMCs respectively.

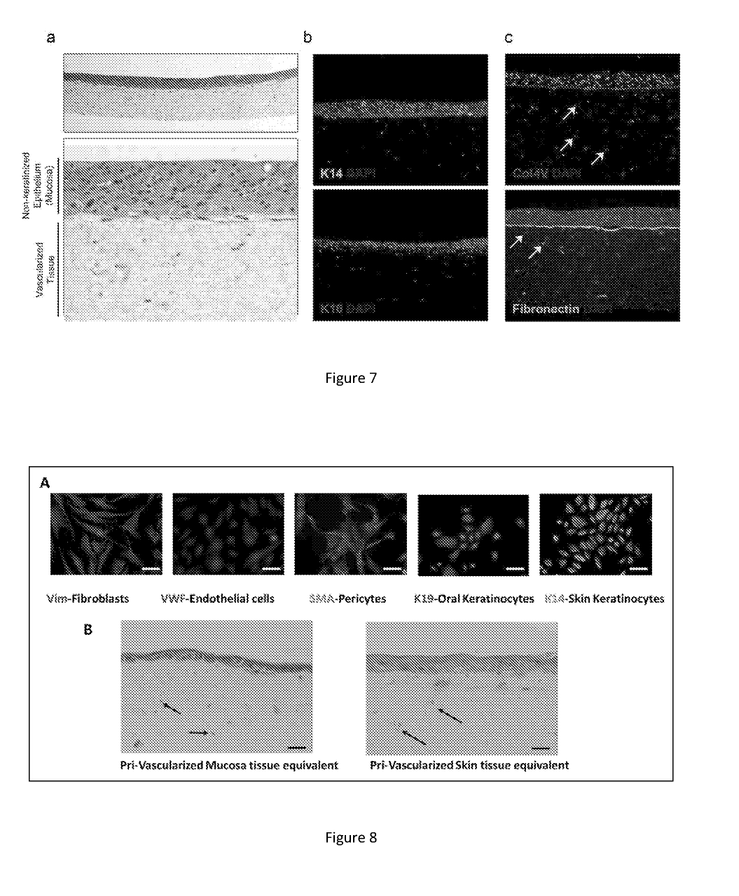

FIG. 7: (a) Representative photomicrographs of haematoxylin and eosin (H-E) stained sections of 3D in vitro vascularized mucosa equivalents. The tissue equivalents consists of stratified layers of non-keratinized squamous epithelium and vascularized tissue beneath. The arrows mark the presence of microvasculature. (b) Immunofluorescent photomicrograph of formalin-fixed paraffin-embedded sections of 3D in vitro vascularized mucosa showing the expression of K14 and K10. (c) Immunofluorescent photomicrograph of formalin-fixed paraffin-embedded sections of 3D in vitro vascularized mucosa showing the presence of lumenized microvascular channels (arrows) with expression of collagen type-IV (Col-IV) and fibronectin.

FIG. 8: Shows the immunofluorescence staining (A) Primary cells showing Vimentin in fibroblasts, Von Willebrand Factor (VWF) in endothelial cells, smooth muscle actin (SMA) in smooth muscle cells/pericytes, K19 in oral-keratinocytes and K14 in Skin-keratinocytes. (B) Microscopic images of haematoxylin and Eosin (H&E) stained sections of Pre-Vascularized mucosa and Pre-vascularized Skin tissue equivalents. Tissue equivalents consists of non-keratinized stratified layer (Mucosa model) and Keratinized stratified layer (Skin model). Arrows are representing the presence of blood vessels.

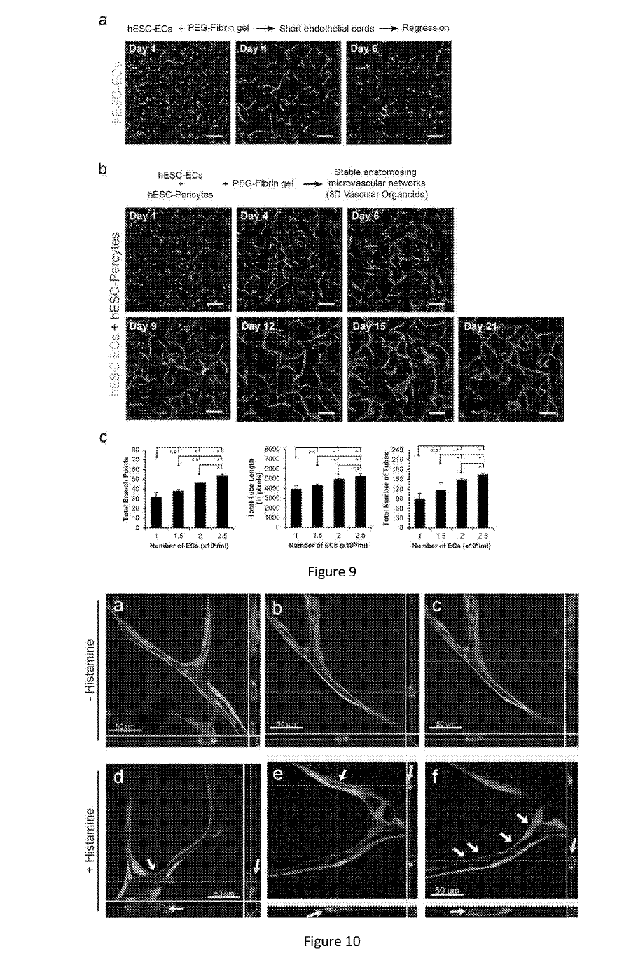

FIG. 9: (a) Representative 3D projection confocal z-stack images of the microvascular networks formed by hESC-ECs (without the hESC-pericytes) after 3D culture in PEG-Fibrin gels for 1, 4 and 6 days. The series of images show the sprouting of ECs that form anastomosing cords after 4 days of culture, but undergo regression after 6 days. (b) Representative 3D projection of confocal z-stack images of the microvascular network formed by hESC-ECs (green) and hESC-pericytes (red) after 3D co-culture in PEG-fibrin gels for 1, 4, 6, 9, 12, 15, and 21 days. The series of images show the sprouting of ECs that forms anastomosing cords after 4-6 days of culture and undergoes maturation in terms of thickness and interconnectivity of the endothelial networks with prolonged culture. Scale bar: 200 μm. (c) Bar charts demonstrate the changes in vascular parameters with changes in seeding density of hESC-ECs. Error bars: s.d. (n≥3). *p<0.05.

FIG. 10: Assessment of Vascular Permeability in vitro. (a-c) The microvascular channels are impermeable to the dextran molecules (red) i.e., the dextran molecules are seen outside the vessel wall, and the lumen is clear. (d-f) However, upon preincubation of the vascular channels with histamine, result in permeabilization of the dextran molecule into the lumen (white arrows) of the microvascular channels, indicating the leakiness in response to histamine. The cross-sectional view of the microvessels shows the presence of the dextran within the lumen (yellow arrows). Scale bar: 50 μm.

### MATERIALS AND METHODS

1. Human embryonic stem cell (hESC) propagation: hESC cell lines were cultured on Matrigel-coated tissue culture plates in complete mTeSR™ 1 medium. Cell lines were characterized routinely for the expression of pluripotentcy markers OCT4, SSEA4 and alkaline phosphatase. Every 5-7 days, cells were passaged by exposing to 1 mg/ml dispase for 5-10 minutes at 37° C. hESC colonies were harvested and broken down to small pieces of colonies by gentle pipetting and plated onto a Matrigel pre-coated plate at 5-6 colonies per 10 cm².

2. Differentiation of hESCs to hESC-KCs: hESCs were propagated as described above. Differentiation of hESCs to hESC-KCs was performed under serum-free media conditions. Keratinocytes differentiation media was prepared with the cocktail of BMP4 (10-50 ng/ml typically 25 ng/ml), retinoic acid (0.1 to 1 uM typically 0.5 μM) and ascorbic acid (10-100 ug/ml typically 50 μg/ml) in defined keratinocyte serum-free medium (DKSFM). Differentiation media was supplied for first 48-96 hours typically 48 hours of differentiation during which neuro-ectoderm lineages were inhibited, after which media was renewed with freshly prepared differentiation media without BMP4. Differentiation process was continued for next 7 to 8 days, with renewing media once in every 48 hours^{1,2}. Once the confluence was reached to 80%, cells were split into 1:3 ratio and seeded onto type-IV collagen (0.5 to 2 ug/cm² typically 1 μg/cm²) or 0.1% gelatin coated plates. Cells were cultured and propagated using DKSFM. After 2-4 passages, matured keratinocytes (hESC-KCs) were characterized by immuno-fluorescence staining and used for further functional studies.

3. Differentiation of hESCs to hESC-oralKCs: hESCs were propagated as described above. Differentiation of hESCs to hESC-oralKCs was performed under serum-free media conditions. Keratinocytes differentiation media was prepared with the cocktail of retinoic acid (0.1 to 2 μM typically 1 μM) and ascorbic acid (10-100 μg/ml typically 50 μg/ml) in DKSFM. Differentiation media was supplied for first 48-72 hers typically 48 hours of differentiation during which neuro-ectoderm lineages were inhibited, after which media was renewed with freshly prepared keratinocyte differentiation media with retinoic acid (0.1 to 2 μM typically 0.5 μM) and ascorbic acid (10-100 μg/ml typically 50 μg/ml). Differentiation process was continued for next 7 to 8 days, with renewing media once in every 48 hours^{1,2}. After 10 days of differentiation, the cells were sorted flow cytometry assisted sorting (FACS) α6-integrin^{high} and CD71^{low} population of cells. The sorted population of α6-integrin^{high} and CD71^{low} cells was seeded onto type-IV collagen (1 μg/cm²). Cells were cultured in DKSFM and propagated on type-IV collagen (1 μg/cm²) or 0.1% gelatin coated plates. After 2-4 passages, matured oral keratinocytes (hESC-oralKCs) were characterized by real-time PCR, immuno-fluorescence staining and used for further functional studies.

4. Differentiation of hESCs to fibroblasts: hESCs were differentiated to hESC-Fib as previously described by our group^{3,4}.

5. Differentiation of hESCs to vascular cells: hESCs propagated under feeder-free conditions were seeded on fibronectin pre-coated plates. 24 hours was allowed for hESCs colonies to attach. After which culture medium was changed to STEMdiff™ APEL medium (a chemically-defined, animal-component free medium). hESCs were directed towards primitive streak by inhibiting GSK-3 (glycogen synthase kinase-3) pathway using BIO/CHIR 98014 or CHIR99021 (2-6 μM typically 5 μM) resulting in down-regulation of pluripotency and ectodermal markers. Subsequently, differentiation was carried by treating cells with basic fibroblast growth factor (bFGF 10-100 ng/ml) typically at 50 ng/ml for 24 hours, after which cells were incubated with VEGF (10-100 ng/ml typically 50 ng/ml) for 72 hours. On day 5 of differentiation, cells were FACS sorted for CD34+CD31+ cells (hESC-endothelial progenitors) and for PDGFβ+CD34-CD31-cells (hESC-vSMC progenitors). FACS sorted hESC-endothelial progenitors were seeded on fibronectin pre-coated plates (1-5 μg/cm2 typically 1.5 μg/cm²) and cultured in endothelial serum-free media (ESFM, GIBCO) supplemented with VEGF (20 to 25 ng/ml typically 0 ng/ml), bFGF (0-50 ng/ml typically 10 ng/ml) and EGF (0-20 ng/ml typically 5 ng/ml) for 2 to 5 passages. Similarly, the hESC-vSMC progenitor cells were FACS sorted, seeded on fibronectin pre-coated plates (1-5 μg/cm² typically 1.5 μg/cm²) and cultured in smooth muscle cell serum-free medium supplemented with PDGFbb (1-20 ng/ml typically 10 ng/ml), bFGF (0-20 ng/ml typically 10 ng/ml) and EGF (0-20 ng/ml typically 5 ng/ml) for 3 to 10 passages^{5,6}. After 2-4 passages of culture, hESC-ECs and hESC-vSMCs were characterized for expression of endothelial and vSMC markers respectively and used for functional studies. The in vitro functionality of hESC-ECs was investigated using Matrigel tube formation assay as previously published by our group⁵.

6. Fabrication of PEG-fibrin gels: Polyethylene-glycol (PEG)-Fibrin gel was fabricated by modification of a published protocol′. Fibrinogen from human or bovine plasma, PEG-4-arm succinimidyl glutarate terminated, thrombin and calcium chloride were used. Working stocks of all the four chemicals were prepared by following manufacturer's instructions. Briefly, fibrinogen was reconstituted at a concentration of 80 mg/ml in 0.1 M sodium bicarbonate (pH-8.3) and mixed by gentle shaking for 1 hour at room temperature and stocks were stored at −80° C. after aliquoting. PEG was reconstituted at a concentration of 8 mg/ml and aliquots stored at −20° C. Human or bovine thrombin was aliquoted at concentration of 100 U/ml and stored at −20° C. Scaffolds were fabricated by mixing the PEG-Fibrinogen at ratio of 10:1 to 50:1 typically 40:1, considering the final concentration of fibrinogen and PEG to 10 mg/ml and 0.25 mg/ml, respectively. This mixture was incubated at 37° C. for 20 to 30 minutes. Thrombin and CaCl₂ (40 mM) were mixed in ratio of 1:3, respectively and placed on ice for 20 to 30 minutes. Various cell types needed are added to PEG-Fibrinogen solution. Equal volumes of Thrombin-CaCl₂ and PEG-fibrinogen-cell suspension were mixed for fabrication of vascularized dermal equivalent. After 10 minutes of incubation at 37° C., 3D cell scaffolds were nourished with 3D vascularized skin media.

7. 3D-Vascularized Skin Media:

Considering the different culture stages, culture media is divided into three different medium.

A. 3D Vascularization Media: consists of serum free Endothelial media as basal media to which vascular growth supplements like vascular endothelial growth factor (VEGF, 5-50 ng/ml typically 20 ng/ml), basic fibroblast growth factor (bFGF 1-25 ng/ml typically 20 ng/ml) and epidermal growth factor (EGF, 1-20 ng/ml typically 10 ng/ml) were added along with antibiotics. Aprotinin (25-200 KIU/ml typically 100 KIU/ml) is also included which inhibits the fibrin degradation.

B. 3D Epithelial Media: This media was added to cultures upon seeding hESC-KCs on top of vascularized dermal equivalents. This media consists of serum free endothelial media with VEGF (5-50 ng/ml typically 20 ng/ml), bFGF (1-25 ng/ml typically 20 ng/ml), EGF (1-20 ng/ml typically 10 ng/ml), aprotinin (25-200 KIU/ml typically 100 KIU/ml), ascorbic acid (10-100 ug/ml typically 50 μg/ml), insulin (5-20 ug/ml typically 10 μg/ml), selenium (1-10 ug/ml typically 5 μg/ml), transferrin (1-10 ug/ml typically 5.5 μg/ml) and antibiotics.

C. 3D Cornification Media: This media was used for culture of the vascularized skin equivalent at air-liquid interphase. This media consists of serum free endothelial media with VEGF (5-50 ng/ml typically 20 ng/ml), bFGF (1-25 ng/ml typically 20 ng/ml), EGF (1-20 ng/ml typically 10 ng/ml), Aprotinin (25-200 KIU/ml typically 100 KIU/ml), ascorbic acid (10-100 μg/ml typically 50 μg/ml), insulin (5-20 μg/ml typically 10 μg/ml), selenium (1-10 μg/ml typically 5 μg/ml), transferrin (1-10 μg/ml typically 5.5 μg/ml), CaCl2 (1-1.8 mM typically 1.2 mM), hydrocortisone 0.1-1 μg/ml typically (0.5 μg/ml), tri-iodo L-thyronine (1-5 nM typically 2 nM), and antibiotics.

8. Formation of In-Vitro 3D Vascularized Skin/Mucosa:

3D in-vitro constructs were developed by considering the PEG-Fibrin hydrogels as scaffolds which acts as platform for cells to grow in and on it. The in vitro vascularized skin equivalents were fabricated by sequentially developing the vascularized dermal equivalent followed by epidermis. The vascularized dermal equivalent was fabricated by encapsulating the hESC-ECs (1-5×10⁶ typically 2.5×10⁶ hESC-ECs/mL of PEG-fibrin gel), hESC-vSMCs and hESC-Fib (in a ratio of 10:1:1 to 40:1:1 with concentration of ECs ranging between 1-5×10⁶ hESC-ECs/mL typically a ratio of 20:1:1) in PEG-fibrin gel. Briefly, fibrinogen from human or bovine plasma, PEG-4-arm succinimidyl glutarate terminated, human thrombin and calcium chloride were used. Working stocks of all the four chemicals were prepared by following manufacturer's instructions. Fibrinogen was reconstituted at a concentration of 80 mg/ml in 0.1 M sodium bicarbonate (pH-8.3), mixed by gentle shaking for 1 hour at room temperature and stocks were stored at −80° C. after aliquoting. PEG was reconstituted at a concentration of 8 mg/ml and aliquots stored at −20° C. Human or bovine thrombin was reconstituted at concentration of 100 U/ml in sterile distilled water and aliquots stored at −20° C. Scaffolds were fabricated by mixing the PEG-Fibrinogen at a ratio of 10:1 to 50:1 with the concentration of fibrinogen fixed at 10 mg/ml typically at a ratio of 40:1, considering the final concentration of fibrinogen and PEG to 10 mg/ml and 0.25 mg/ml, respectively. This mixture was incubated at 37° C. for 20 to 30 minutes. Thrombin (100 U/ml) and CaCl₂ (40 mM) were mixed in ratio of 1:3, respectively and placed on ice. The cells (hESC-ECs, hESC-vSMCs and hESC-Fib) were suspended in 100 μl of PEG-fibrinogen solution and mixed with 100 μl of thrombin-calcium chloride solution, immediately pipetted into a 12-well culture insert to form a PEG-fibrin gel that upon culture results in the formation of vascularized dermal equivalent. The PEG-fibrin with the hESC-ECs, hESC-vSMCs and hESC-Fib were nourished with 3D vascularization media (described above) for 10 days with media changes every 24 hours. After the 10-day 3D tri-culture period the keratinocytes were seeded on top of the vascularized dermal equivalent at a seeding density of 10 to 40×10⁴/cm² typically 25×10⁴ cells/cm². For generation of in vitro vascularized skin equivalents, hESC-KCs were seeded, while for the generation of in vitro vascularized mucosa equivalents, hESC-oralKCs were seeded. In this phase of keratinocyte culture, the PEG-fibrin gels were nourished with 3D epithelial media for 2-3 days with media renewal every 24 hours. Then, the 3D co-cultures were cultured at air-liquid interface by transferring the culture inserts to a 12-well deep well plate (Griener BioOne) and media supply from only the bottom surface (while the top surface was exposed to air). The media used at this phase was 4 mL/well of 3D cornification media. At the end of third weeks of culture at air-liquid interphase, the 3D cultures were fixed overnight using 4% paraformaldehyde (PFA) at 4° C. and paraffin-embedded. Sections of formalin-fixed paraffin-embedded samples were used for routine histological analysis using haematoxylin-eosin staining and immunofluorescence staining for vascular markers and epithelial markers. Similarly, in a separate experimental setup, PEG-Fibrin scaffolds were fabricated with primary cells viz, endothelial, pericytes, fibroblasts, dermal keratinocytes and oral keratinocytes to form 3D vascularized skin/mucosa, considering primary cell based models as the control 3D skin/mucosa models (depicted in FIG. 8).

Results

1. Culture and Characterization of hESCs:

The hESCs cultured on Matrigel were routinely characterized for pluripotency markers as depicted in FIG. 1.

2. Differentiation of hESCs to hESC-KCs:

hESCs were differentiated to hESC-KCs as described above and depicted in FIG. 2. Sequential treatment of hESCs grown on Matrigel-coated plates in DKSFM with BMP4, retinoic acid (RA) and ascorbic acid (AA) as depicted in FIG. 2a, resulted in emergence of colonies of hESC-derived epithelial progenitors. Dissociation of the hESC-derived epithelial progenitors and serial passage onto collagen type-IV or gelatin-coated plates resulted in the maturation of the hESC-epithelial progenitors to hESC-KCs (FIG. 2b). These hESC-KCs were positive for basal keratinocyte markers K14 and p63 confirming the identity of the keratinocyte lineage (FIG. 2c).

3. Differentiation of hESCs to hESC-oralKCs:

hESCs were differentiated to hESC-KCs as described above and depicted in FIG. 3. Sequential treatment of hESCs grown on Matrigel-coated plates in DKSFM with retinoic acid (RA) and ascorbic acid (AA) as depicted in FIG. 3a, resulted in emergence of colonies of hESC-derived epithelial progenitors. These progenitors were FACS sorted for α6-integrin^{high} and CD71^{low} population, seeded on to collagen type-IV coated plates and cultured in DKSFM. Serial passage onto collagen type-IV or gelatin-coated plates resulted in the maturation to hESC-oralKCs (FIG. 3b). These hESC-oralKCs were positive for basal keratinocyte markers K14 and p63 confirming the identity of the keratinocyte lineage (FIG. 3c).

4. Differentiation of hESCs to hESC-ECs:

hESCs were differentiated to hESC-ECs as depicted in FIG. 4. We had earlier established a novel protocol to efficiently drive the differentiation of hESCs to primitive streak-like stage (PS) through short-term inhibition of glycogen synthase kinase-3β (GSK3) which could be induced to lateral and paraxial mesoderm subtypes through modulation of BMP4 and VEGF 6. We modified our earlier protocol by differentiation of hESCs over human plasma fibronectin as substrate (instead of Matrigel) and driving the differentiation of hESC-derived PS cells (24 hours of GSK3 inhibition using CHIR99021) towards mesoderm through a short-term bFGF pulse (24 hours) before induction to vascular lineage (a lateral plate mesoderm derivative) using VEGF as outlined in FIG. 4a. After 5 days of differentiation, the CD34+CD31+ cells (hESC-endothelial progenitors) were FACS sorted and seeded onto fibronectin coated plates and further differentiated to hESC-ECs in ESFM supplemented with VEGF, bFGF and EGF (FIG. 4b-c). The terminally differentiated cells attained cobble-stone morphology, expressed endothelial markers CD31, VE-Cadherin and von Willebrand factor (vWF) (FIG. 4d-h). Additionally, the ECs showed the ability to self-organize to form vascular cord-like structures over Matrigel (FIG. 4h). In summary, these findings indicate the differentiation of hESCs to hESC-ECs under feeder-free and serum-free conditions.

5. Differentiation of hESCs to hESC-vSMCs:

hESCs were differentiated to hESC-vSMCs (or hESC-Pericytes) as depicted in FIG. 5. hESCs were differentiated towards vascular lineage through sequential treatment with CHIR99021 (5 μM), bFGF and VEGF as outlined in FIG. 5a. After 5 days of differentiation, the PDGFRβ+CD34-CD31 cells (hESC-paraxial mesoderm progenitors) were FACS sorted (FIG. 5b-c) and seeded onto fibronectin coated plates and further differentiated to hESC-vSMCs/Pericytes in SFM supplemented with PDGFbb, bFGF and EGF. The terminally differentiated cells attained spindle-shaped morphology, expressed vSMC markers alpha smooth muscle actin (αSMA) and calponin (CNN1) (FIG. 5d-i). In summary, these findings indicate the differentiation of hESCs to hESC-vSMCs (or hESC-Pericytes) under feeder-free and serum-free conditions.

6. Fabrication of 3D In Vitro Vascularized Skin Equivalent:

As mentioned in the methods section, 3D in vitro vascularized skin equivalent was fabricated by sequentially developing the vascularized dermal equivalent followed by epidermis. The vascularized dermal equivalent was fabricated by encapsulating hESC-ECs, hESC-vSMCs and hESC-Fib within PEG-fibrin gel as scaffold. Then, the vascularized dermal equivalent was epithelialized by seeding hESC-KCs and cultured at air-liquid interface. After 3 weeks of culture at air-liquid interface, the 3D co-cultures were formalin-fixed and embedded in paraffin. Haematoxylin and eosin (H-E) stained cross-sections showed the presence of epidermis and dermis. The epidermis consisted of stratified layers of keratinocytes and cornification, while the dermis showed the presence of microvasculature and fibroblasts (FIG. 6a). Immunofluorescent staining of formalin-fixed paraffin-embedded cross-sections of 3D in vitro vascularized skin equivalents showed the expression of K14 (FIG. 6b). To visualize the presence of vasculature, the 3D in vitro vascularized skin equivalents were sectioned transversely on the dermal side. H-E staining of these transverse sections showed the presence of interconnecting network of microvascular channels (FIG. 6c). Further, immunofluorescent staining of these transverse sections showed the presence of vWF-expressing hESC-ECs along the periphery of the microvascular channel and calponin (CNN1) expressing hESC-vSMCs outside the microvascular channels in the extracellular matrix of the dermis (FIG. 6d).

7. Fabrication of 3D In Vitro Vascularized Mucosa Equivalent:

As mentioned in the methods section, 3D in vitro vascularized mucosa equivalent was fabricated by sequentially developing the vascularized tissue equivalent followed by mucosal epithelium. The vascularized tissue equivalent was fabricated by encapsulating hESC-ECs, hESC-vSMCs and hESC-Fib within PEG-fibrin gel as scaffold as described above. Then, the vascularized tissue equivalent was epithelialized by seeding hESC-oralKCs and cultured at air-liquid interface. After 3 weeks of culture at air-liquid interface, the 3D co-cultures were formalin-fixed and embedded in paraffin. Haematoxylin and eosin (H-E) stained cross-sections showed the presence of non-keratinized stratified squamous epithelium representative of oral mucosa. The tissue beneath the epithelium shows the presence of microvasculature and fibroblasts (FIG. 7a). Immunofluorescent staining of formalin-fixed paraffin-embedded cross-sections of 3D in vitro vascularized mucosa equivalents showed the expression of K14 and K10 (FIG. 7b). To visualize the presence of vasculature, the 3D in vitro vascularized mucosa equivalents were immunostained for basement membrane markers collagen type-IV and fibronectin. The immunofluorescent staining showed the expression of collagen-IV and fibronectin along the walls of microvascular channels and at the junction of epithelium and the sub-epithelial tissue (FIG. 7c).

8. Primary Cell Lines Based Models:

FIG. 8 (A) represents the immunofluorescence staining of monolayers of primary cells, highlighting the expression of Vimentin in fibroblasts, Von Willebrand Factor (VWF) in endothelial cells, smooth muscle actin (SMA) in smooth muscle cells/pericytes, K19 in oral-keratinocytes and K14 in Skin-keratinocytes. FIG. 8 (B) represents the microscopic images of haematoxylin and Eosin (H&E) stained sections of Pre-Vascularized mucosa and Pre-vascularized Skin tissue equivalents. Tissue equivalents consists of non-keratinized stratified layer (Mucosa model) and Keratinized stratified layer (Skin model). Arrows are representing the presence of blood vessels showing the tissue is vascularised.

9.

### Example-1: In Vitro Vascularized Tissue Equivalents as Model to Study Endothelial Regression

In vascular development, absence of recruitment of mural cells (pericytes) is associated with regression of early endothelial vessels⁹. To investigate and model endothelial regression, we cultured hESC-ECs (eGFP labelled) alone within PEG-Fibrin gels. This was associated with the following morphological changes (FIG. 9a). After 1 day of culture most of the hESC-ECs are primarily rounded, while a small percentage of the ECs displayed elongated cytoplasm indicating endothelial sprouting. Though, the hESC-ECs formed short anastomosing cords of ECs through intercellular organization after 4 days of culture, by 6th day of culture the endothelial cords started decreasing in number, length and complexity to few small endothelial cords and rounded cells (FIG. 9b). By 8th-9th day of culture, no cells were visible for visualization by confocal microscopy indicating the lack of hESC-ECs to sustain the formation of vascular channels and demonstrate regression of endothelial cords.

Hence, this in vitro human vascularized tissue equivalent model paves way to study endothelial regression observed in embryonic development and tumour angiogenesis.

### Example-2: Demonstration of Kinetics of Vascular Development

Recruitment of mural cells to developing endothelial vessels is known to be critical for the formation, maturation and stabilization of vascular networks⁹. In order to study the kinetics of vascular development, hESC-ECs (eGFP labelled) were co-cultured with hESC-pericytes (DsRed2-labelled) within PEG-Fibrin gels and imaged over 3 weeks using confocal microscopy. In the co-culture gels, the hESC-ECs formed robust microvascular networks that start as few elongated endothelial cords by 4th day followed by an apparent increase in number, length, branches, anastomoses and complexity with increasing days of culture (FIG. 9c). Further, these bicellular microvascular networks had evidence of almost continuous, connected lumen formation and were stable in culture for 3 weeks (FIG. 9b).

Hence, this in vitro human vascularized tissue equivalent model paves way to study kinetics of vascular development. Further, it can be used to study to effect of drugs (inhibitors/stimulators) targeting angiogenesis on the kinetics of vascular development and morphogenesis. Taken together, these findings establish the utility of 3D in vitro vascularized tissue equivalents as an in vitro model for quantitative and qualitative assessment of fractal dimensions of the microvascular network. The in vitro 3D vascular organoids could potentially be employed as a physiological 3D model of tissue microvasculature for high-throughput screening of novel pro- and anti-angiogenesis compounds in vitro.

### Example-3: Investigating the Effect of Endothelial Cells on Vascular Development

We also analyzed the effect of endothelial cells on vascular morphogenesis by altering the seeding density of hESC-ECs while keeping the ratio of hESC-ECs to hESC-Pericytes constant (20:1). The ratio of ECs to vSMCs/pericytes is reported to vary from 1:1 to 100:1 depending on the tissue in the body¹⁰. In this study, we used a fixed ratio of 20:1 (ECs to pericytes) for all the experiments. The hESC-ECs formed anastomosing network of organotypic microvascular channels within about 6 days. Depending on the initial seeding density of hESC-ECs, the microvascular structures extended, branched and anastomosed into networks. Various parameters related to microvascular networks that included total length of the vascular network, total number of tubes and the number of branching points within the network were used to narrow down on the optimal density of hESC-ECs for further experiments. Endothelial seeding density studies showed a significant increase in the total tube length, number of tubes, and number of branching points with increase in the initial seeding density of hESC-ECs (FIG. 9c). At concentrations above 3×106 hESC-ECs/mL, the hESC-ECs formed numerous, long thin cords but did not survive after 4 days of culture; and the matrix showed signs of disintegration. These observations might obviously be due to competition for growth factors and nutrients, and also due to excessive remodeling of the matrix by the hESC-vascular cells. On the other hand, at low concentrations (<100,000 cells/mL), only focal outgrowth of vascular structures restricted to certain regions within the whole matrix were observed.

Overall, these results demonstrate the ability to study human vascular development in vitro using these in vitro vascularized tissue equivalent models. These applications demonstrate the ability of these vascularized tissue equivalents as a novel in vitro tool for testing drugs (inhibitors and stimulators) targeting angiogenesis.

### Example-4: Investigating Vascular Permeability

An important role of ECs is to maintain a tight dynamic barrier to regulate the transport of fluids, molecules and cells between the intraluminal and extraluminal compartments of the blood vessels. Monolayer of ECs are relatively impermeable to macromolecules (1-100 kDa) with <1% flux¹¹. To assess the permeability of the implanted microvessels in-vivo, studies use fluorescent tracers and/or non-invasive live imaging¹². In-vitro equivalent of permeability testing, typically measures the transendothelial resistance across a 2D monolayer of ECs (without the presence of supporting mural cells) in a transwell system¹³. Alternatively, the permeation of fluorescently/radioisotope labeled chemicals could be used to assess the movement of the chemicals across the endothelial monolayer.

10. As a proof of concept to assess the permeability of vascular channels within the 3D vascularized tissue equivalents, we utilized a principle of inverse permeability. The principle of inverse permeability is that mature microvessels are impermeable to dextrans over a molecular weight of 65 kDa, and a tracer would be able to enter inside the lumen of leaky vascular channels, while it cannot enter inside a vascular channel with mature, competent cell-cell endothelial junctions. Endothelial permeability to macromolecules increases markedly upon exposure to variety of compounds like histamine, prostaglandin E2, spingosine-2-phosphate and cyclic adrenomedullin. We adapted the method of inverse permeability to assess the barrier properties of the microvascular networks within hESC-derived in-vitro 3D vascularized tissue equivalents. Dextran conjugated to Texas Red (70 kDa) was used as the tracer dye to assess the permeability of the microvessels.

Confocal imaging of the 3D constructs after incubation with the tracer dye revealed that most of the microvessels were impermeable to the dye as demonstrated by the restriction of the red tracer dye to the extravascular space (outside the blood vessel) (FIG. 10a-c). On the other hand, pre-incubation of the constructs with histamine resulted in marked increase in the permeability of the microvascular channels as evidenced by the presence of aggregates of the tracer dye within the vascular lumen (FIG. 10d-f). The impermeability of microvascular channels to the tracer dye and an increased leakiness in response to physiological stimulus like histamine, also reveal the maturity and functionality of the 3D in vitro vascularized tissue equivalents.

Taken together, these findings establish the utility of 3D in vitro vascularized tissue equivalents as an in vitro model for qualitative assessment of vascular permeability and could potentially be employed as a physiological 3D model of tissue microvasculature for high-throughput screening of vascular drugs.

### CONCLUSION

In conclusion, using co-culture of four different cell types differentiated from a single source (hESCs) within PEG-fibrin gel we have demonstrated the ability to fabricate 3D in vitro vascularized skin and mucosa equivalents. We are the first to develop a 3D in vitro vascularized skin and mucosa equivalent of hESC origin. Secondly, we are the first to demonstrate the ability to culture four different cell types needed for generation of 3D in vitro vascularized skin and mucosa equivalent. Additionally, we have compared our model with primary cell lines based models, which proves hESC based 3D tissue equivalents are more reliable and provides acceptable tissue physiology. We strongly believe that this technology could be simulated with primary cells, human adult stem cells, and induced pluripotent stem cells.

### REFERENCES

1. Kidwai, F. K. et al. Differentiation of human embryonic stem cells into clinically amenable keratinocytes in an autogenic environment. The Journal of investigative dermatology 133, 618-628 (2013).
2. M. Movahednia, F. K., Y. Zou, H. J. Tong, X. Liu, I. Islam, W. S. Toh, M. Raghunath, T. Cao, Differential effects of the extracellular microenvironment on human embryonic stem cells differentiation into keratinocytes and their subsequent replicative lifespan. Tissue Engineering Part A, (In press). (2015).
3. Fu, X. et al. Establishment of clinically compliant human embryonic stem cells in an autologous feeder-free system. Tissue engineering. Part C, Methods 17, 927-937 (2011).
4. Fu, X. et al. Autologous feeder cells from embryoid body outgrowth support the long-term growth of human embryonic stem cells more effectively than those from direct differentiation. Tissue engineering. Part C, Methods 16, 719-733 (2010).
5. Tan, J. Y., Sriram, G., Rufaihah, A. J., Neoh, K. G. & Cao, T. Efficient derivation of lateral plate and paraxial mesoderm subtypes from human embryonic stem cells through GSKi-mediated differentiation. Stem cells and development 22, 1893-1906 (2013).
6. Sriram, G. In-vitro vacularized tissue equivalents from human embryonic stem cell-derived endothelial and smooth muscle cells. (2014).
7. Natesan, S., Zamora, D. O., Suggs, L. J. & Christy, R. J. Engineering a bilayered hydrogel to control ASC differentiation. Journal of visualized experiments: JoVE, e3953 (2012).
9. Jain, R. K. Molecular regulation of vessel maturation. Nature medicine 9, 685-693 (2003).
10. Shepro, D. & Morel, N. M. Pericyte physiology. FASEB journal: official publication of the Federation of American Societies for Experimental Biology 7, 1031-1038 (1993).
11. Glaser, D. E. et al. Functional characterization of embryonic stem cell-derived endothelial cells. J Vasc Res 48, 415-428 (2011).
12. Samuel, R. et al. Generation of functionally competent and durable engineered blood vessels from human induced pluripotent stem cells. Proc Natl Acad Sci USA 110, 12774-12779 (2013).
13. Adams, W. J. et al. Functional vascular endothelium derived from human induced pluripotent stem cells. Stem cell reports 1, 105-113 (2013).

1. A method for the preparation of an organotypic vascularized tissue, skin or mucosa equivalent or composition comprising the steps of:
i) obtaining a preparation of mammalian pluripotent stem cells and culturing the cells under cell culture conditions to induce the formation of the following differentiated cell types: endothelial cells (SC-ECs), vascular smooth muscle cells/pericytes (SC-vSMCs), fibroblasts (SC-Fib) and keratinocytes (SC-KCs);
ii) seeding the SC-ECs, SC-vSMCs and, optionally, SC-Fib of part i) in or on a scaffold and further culturing the cells under cell culture conditions to induce the formation of a vascularized dermal layer; and
iii) seeding the SC-KCs of part i) onto the vascularized dermal layer of part ii) and further culturing the cells under cell culture conditions to induce the formation of a stratified layer of keratinized epidermis upon said vascularized dermal layer to provide an organotypic vascularized skin or mucosa equivalent.

2. The method according to claim 1, wherein said mammalian pluripotent stem cells are human.

3. The method according to claim 1, wherein said mammalian pluripotent stem cells are human embryonic stem cells (hESC) or human embryonic germ cells (hEGC) or human induced pluripotent stem cells (hiPSC).

4. The method according to claim 1, wherein said cell culture conditions comprise additional cell types such as but not limited to melanocytes or macrophages.

5. The method according to claim 4, wherein said additional cell types are derived from human embryonic stem cells (hESC), human embryonic germ cells (hEGC), or human induced pluripotent stem cells (hiPSC).

6. The method according to claim 1, wherein said mammalian endothelial cells (SC-ECs), vascular smooth muscle cells (SC-vSMCs), fibroblasts (SC-Fib) and keratinocytes (SC-KCs) are autologous.

7. The method according to claim 1, wherein said mammalian endothelial cells (SC-ECs), vascular smooth muscle cells (SC-vSMCs), fibroblasts (SC-Fib) and keratinocytes (SC-KCs) are allogeneic.

8. The method according to claim 1, wherein said method comprises culturing said cells in step ii) for at least 1-20 days, including all one day intervals in between, prior to undertaking step iii).

9. The method according to claim 1, wherein said scaffold comprises a natural or hybrid polymer based scaffold such as but not limited to polyethylene glycol-fibrin, fibrin, collagen type-1, hyaluronic acid gel scaffold, or the like.

10. The method according to claim 1, wherein said scaffold comprises a biocompatible polymer based scaffold such as but not limited to a polyester including polystyrene, polylactic acid, polyglycolic acid, polycaprolactone, poly-dl-lactic-co-glycolic acid, or the like.

11. The method according to claim 1, wherein said hESC-ECs, hESC-vSMCs and hESC-Fib are provided in said scaffold in a ratio of about 10:1:1 to about 40:1:1.

12. The method according to claim 1, wherein said mammalian keratinocytes are cultured at an Air-Liquid Interface.

13. The method according to claim 1, wherein said Keratinocytes are seeded on top of the vascularized dermal layer at a seeding density of about 10×10⁴ to about 40×10⁴ cells/cm².

14. The method according to claim 1, wherein said Keratinocytes are either hESC-KCs, for the generation of in vitro vascularized skin equivalent, or hESC-oralKCs for the generation of in vitro vascularized mucosa equivalent.

15. The method according to claim 1, wherein said cell culture conditions comprises serum free media.

16. The method according to claim 1, wherein said organotypic vascularized tissue, skin or mucosa equivalent prepared by the steps of i)-iii) is maintained in cell culture.

17. An isolated differentiated mammalian endothelial cell (hESC-ECs), vascular smooth muscle cell/pericyte (hESC-vSMCs), fibroblast (hESC-Fib) or keratinocyte (hESC-KCs) obtained or obtainable by the method according to claim 1.

18. An organotypic vascularized tissue, skin or mucosa equivalent or composition obtained or obtainable by the method according to claim 1.

19. A therapeutic tissue/skin graft or implant comprising an organotypic tissue or skin composition obtained or obtainable by the method according to claim 1.

20. The therapeutic tissue/skin graft or implant according to claim 19 for use in the treatment of skin damage.

21. The therapeutic tissue/skin graft or implant according to claim 20 for use in the treatment of skin damage as a result of: infection or trauma, including wounding, scarring, or burns, or in response to disease such as skin grafts required as a consequence of tissue removal in cancer or in the treatment of diabetic or non-diabetic ulcers.

22. A cosmetic tissue/skin graft or implant comprising an organotypic skin composition obtained or obtainable by the method according to claim 1.

23. A method of treatment comprising administering or implanting a tissue/skin graft or implant according to claim 19 at or into a site of a mammal to be treated.

24. A method of cosmetic surgery comprising implanting a tissue/skin graft or implant according to claim 22 at or into a site of a mammal to be treated.

25. A cell culture vessel comprising an organotypic tissue, skin or mucosa equivalent or composition according to claim 18.

26. The cell culture vessel according to claim 25 wherein said cell culture vessel comprises a cell culture insert, optionally removable, containing said organotypic tissue, skin or mucosa equivalent in fluid contact with cell culture medium.

27. An organotypic vascularized tissue, skin or mucosa equivalent or composition according to claim 18 for use in the testing of test agents such as but not limited to therapeutics, cosmetics, compounds or biologically active xenobiotic agents.

28. A cell array wherein said array comprises a plurality of cell culture vessels according to claim 25.

29. A method for the high throughput screening of test agents comprising the steps:
i) providing an array according to claim 28;
ii) contacting the array with a plurality of agents to be tested;
iii) collating activity data obtained following (ii) above;
iv) converting the collated data into a data analyzable form; and optionally
v) providing an output for the analysed data.

30. A method for the preparation of an organotypic tissue, skin or mucosa equivalent or composition comprising the steps:
i) seeding endothelial cells and vascular smooth muscle cells and, optionally, fibroblasts in or on a scaffold to provide a vascularized dermal layer; and
ii) seeding keratinocytes onto the vascularized dermal layer of part i) and further culturing the cells under cell culture conditions to induce the formation of a stratified layer of keratinized epidermis upon said vascularized dermal layer to provide an organotypic tissue, skin or mucosa equivalent.

31. The method according to claim 30, wherein said organotypic tissue, skin or mucosa equivalent prepared by the steps of i)-ii) is maintained in cell culture.
